(19) United States
(12) Patent Application Publication
(10) Pub. No.: US 2026/0240652 A1
(43) Pub. Date: Aug. 20, 2026

(54) LEAFLET MODIFICATION DEVICE WITH RADIOPAQUE ALIGNMENT MARKERS

(71) Applicant: BOSTON SCIENTIFIC SCIMED, INC., MAPLE GROVE, MN (US)

(72) Inventors: James M. Anderson, Corcoran, MN (US); Lauren Koon, Etobicoke (CA); Phil Litecky, Forest Lake, MN (US); Eric Michael Petersen, Maple Grove, MN (US)

(73) Assignee: BOSTON SCIENTIFIC SCIMED, INC., MAPLE GROVE, MN (US)

(21) Appl. No.: 19/541,771

(22) Filed: Feb. 17, 2026

Related U.S. Application Data
(60) Provisional application No. 63/759,364, filed on Feb. 17, 2025.

(51) Int. Cl. A61F 2/24; A61B 18/14

(52) U.S. Cl. CPC A61F 2/2436; A61B 18/1492

(57) ABSTRACT

A valve leaflet resection device includes a catheter (110), an electrode assembly (140) slidably disposed within the catheter and deployable from the catheter and configured to remove a portion of a leaflet of a heart valve, a leaflet retainer (130) slidably disposed within the catheter and deployable from the catheter, the leaflet retainer configured to grasp a leaflet of a heart valve, and a capture basket (120) configured to capture a resected portion of the leaflet of the heart valve upon retraction of the leaflet retainer.

### CROSS REFERENCE TO RELATED APPLICATIONS

This application claims the benefit of priority of U.S. Provisional Application No. 63/759,364 filed February 17, 2025, the entire disclosure of which is hereby incorporated by reference.

### TECHNICAL FIELD

The present disclosure relates generally to medical devices. More particularly, the present disclosure pertains to medical devices for lacerating cardiac valve leaflets.

### BACKGROUND

A wide variety of intracorporeal medical devices have been developed for medical use, and more specifically for intravascular use. Some of these devices include devices for lacerating cardiac valve leaflets. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for lacerating cardiac valve leaflets. An example may be found in a medical device for lacerating valve leaflets. The medical device includes an elongate shaft and an inflatable balloon that is secured to the elongate shaft. The inflatable balloon has an outer surface and includes a deflated configuration and an inflated configuration. An electrocautery electrode is secured relative to the outer surface of the inflatable balloon. One or more radiopaque markers are disposed relative to the electrocautery electrode and are adapted to indicate relative rotational alignment of the electrocautery electrode.

Alternatively or additionally, the one or more radiopaque markers may be disposed on the outer surface of the inflatable balloon.

Alternatively or additionally, the one or more radiopaque markers may be disposed within the inflatable balloon.

Alternatively or additionally, the elongate shaft may include a shaft portion that extends through an interior of the inflatable balloon, and the one or more radiopaque markers may be secured relative to the shaft portion.

Alternatively or additionally, the one or more radiopaque markers may include one or more radiopaque markers that are at least substantially parallel with the electrocautery electrode.

Alternatively or additionally, the one or more radiopaque markers may include one or more radiopaque markers that are at least substantially orthogonal to the electrocautery electrode.

Alternatively or additionally, the one or more radiopaque markers may include one or more radiopaque marker segments that are at least substantially parallel with the electrocautery electrode and one or more radiopaque marker segments that are at least substantially perpendicular to the electrocautery electrode.

Alternatively or additionally, the electrocautery electrode may be adapted to be visible during fluoroscopy.

Alternatively or additionally, the one or more radiopaque markers may include a first radiopaque marker that is secured relative to a first end of the electrocautery electrode and a second radiopaque marker that is secured relative to a second end of the electrocautery electrode.

Alternatively or additionally, the one or more radiopaque markers may include a pair of elongate radiopaque markers that are circumferentially spaced 180 degrees apart from each other.

Alternatively or additionally, the elongate shaft may further include a lumen for injecting contrast media.

Another example may be found in a medical device for lacerating valve leaflets. The medical device includes an inflatable balloon having an outer surface and including a deflated configuration and an inflated configuration. An electrocautery electrode is secured relative to the inflatable balloon. One or more radiopaque markers are secured relative to the inflatable balloon.

Alternatively or additionally, the one or more radiopaque markers may be disposed on the outer surface of the inflatable balloon.

Alternatively or additionally, the one or more radiopaque markers may be at least substantially parallel with the electrocautery electrode.

Alternatively or additionally, the one or more radiopaque markers may be at least substantially orthogonal to the electrocautery electrode.

Alternatively or additionally, some of the one or more radiopaque markers may be at least substantially parallel with the electrocautery electrode and some of the one or more radiopaque marker segments may be at least substantially perpendicular to the electrocautery electrode.

Alternatively or additionally, the one or more radiopaque markers may include a first radiopaque marker that is secured relative to the electrocautery electrode and a second radiopaque that is secured relative to the electrocautery electrode and spaced apart from the first radiopaque marker.

Alternatively or additionally, the one or more radiopaque markers may include a pair of elongate radiopaque markers that are circumferentially spaced 180 degrees apart from each other.

Another example may be found in a medical device for lacerating valve leaflets. The medical device includes an elongate shaft and an inflatable balloon that is secured to the elongate shaft. The inflatable balloon has an outer surface and includes a deflated configuration and an inflated configuration. An electrocautery electrode is secured relative to the outer surface of the inflatable balloon. A radiopaque marker is disposed relative to the electrocautery electrode in order to indicate relative rotational alignment of the electrocautery electrode.

Alternatively or additionally, the medical device may further include a second radiopaque marker.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cutaway view showing an aortic heart valve positioned within a native valve annulus of a heart;

FIG. 2 is a schematic view of an illustrative medical device including radiopaque markers disposed on a shaft of the medical device;

FIG. 3 is a schematic view of an illustrative medical device including radiopaque markers disposed on an inflatable balloon of the illustrative medical device;

FIG. 4A is a schematic view of an illustrative medical device including radiopaque markers indicating an incorrect rotational position relative to the anatomy and FIG. 4B is a schematic view of the illustrative medical device in a correct rotational position;

FIG. 5A is a schematic view of an illustrative medical device including radiopaque markers indicating an incorrect rotational position relative to the anatomy and FIG. 5B is a schematic view of the illustrative medical device in a correct rotational position;

FIG. 6A is a schematic view of an illustrative medical device including radiopaque markers indicating an incorrect rotational position relative to the anatomy and FIG. 6B is a schematic view of the illustrative medical device in a correct rotational position;

FIG. 7 is a schematic view of an illustrative medical device including a lumen for injecting contrast media; and

FIG. 8 is a schematic view of an illustrative medical device including radiopaque markers secured to an electrocautery electrode.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

A number of patients receive artificial heart valves for a variety of reasons including valve malfunction due to calcium accumulation. When an artificial heart valve is implanted, the artificial heart valve may have an expandable frame that presses the native valve leaflets away from the native position of the native valve leaflets. In some instances, the native valve is the aortic valve, and the artificial heart valve is an artificial aortic valve. In some instances, it is possible for one or more of the native valve leaflets, when pressed to the side, to at least partially or even completely block an ostium of one of the coronary arteries. Not only does this present possible health concerns for the patient, particularly if an ostium is completely blocked, but even when an ostium is only partially blocked and thus still permits blood flow, this may present difficulties in subsequently being able to perform balloon angioplasty, or place a stent, in one of the coronary arteries. In some instances, it may be beneficial to slice or lacerate with opportunity to remove one or more of the native valve leaflets prior to implantation of the artificial heart valve so that when the native valve leaflets are pressed to the side by the expandable frame of the artificial heart valve, the native valve leaflets do not block an ostium of any of the coronary arteries.

In some instances, a patient may already have an implanted artificial heart valve such as an artificial aortic valve. The artificial valve leaflets forming part of the already implanted artificial heart valve can be just as problematic with respect to potentially blocking a cardiac artery ostium when displaced to the side when a second artificial heart valve is implanted in place of the first artificial heart valve. The artificial valve leaflets forming part of the artificial heart valve may, for example, be made from porcine or bovine tissue, or may be polymeric. In some instances, artificial valve leaflets may be made of polymers such as Dacron or Gore-Tex. As discussed here, reference to a valve leaflet may refer to either a native valve leaflet or an artificial valve leaflet.

In some instances, a medical device is adapted for lacerating valve leaflets. The medical device includes an elongate shaft and an inflatable balloon that is secured to the elongate shaft. The inflatable balloon has an outer surface and has a deflated configuration and an inflated configuration. An electrocautery electrode is secured relative to the outer surface of the inflatable balloon. One or more radiopaque markers are disposed relative to the electrocautery electrode and are adapted to indicate relative rotational alignment of the electrocautery electrode.

In some cases, the one or more radiopaque markers may be disposed on the outer surface of the inflatable balloon. In some cases, the one or more radiopaque markers may be disposed within the inflatable balloon. The elongate shaft may include a shaft portion extending through an interior of the inflatable balloon and the one or more radiopaque markers may be secured relative to the shaft portion, for example. In some cases, the one or more radiopaque markers may include one or more radiopaque markers that are at least substantially parallel with the electrocautery electrode, where substantially parallel is defined as being within twenty percent of parallel. In some cases, the one or more radiopaque markers may include one or more radiopaque markers that are at least substantially orthogonal to the electrocautery electrode, where substantially orthogonal is defined as being within twenty percent of orthogonal. In some cases, the one or more radiopaque markers may include one or more radiopaque marker segments that are at least substantially parallel with the electrocautery electrode and one or more radiopaque marker segments that are at least substantially perpendicular to the electrocautery electrode.

In some cases, the electrocautery electrode may be adapted to be visible during fluoroscopy. In some cases, the one or more radiopaque markers may include a first radiopaque marker that is secured relative to a first end of the electrocautery electrode and a second radiopaque marker that is secured relative to a second end of the electrocautery electrode. In some cases, the one or more radiopaque markers may include a pair of elongate radiopaque markers that are circumferentially spaced 180 degrees apart from each other. In some cases, the elongate shaft may further include a lumen for injecting contrast media.

In some instances, a medical device is adapted for lacerating valve leaflets. The medical device includes an inflatable balloon that has an outer surface and includes a deflated configuration and an inflated configuration. An electrocautery electrode is secured relative to the inflatable balloon. One or more radiopaque markers are secured relative to the inflatable balloon.

In some cases, the one or more radiopaque markers may be disposed on the outer surface of the inflatable balloon. In some cases, the one or more radiopaque markers may be at least substantially parallel with the electrocautery electrode. In some cases, the one or more radiopaque markers may be at least substantially orthogonal to the electrocautery electrode. In some cases, some of the one or more radiopaque markers may be at least substantially parallel with the electrocautery electrode and some of the one or more radiopaque marker segments may be at least substantially perpendicular to the electrocautery electrode. In some cases, the one or more radiopaque markers may include a first radiopaque marker that is secured relative to the electrocautery electrode and a second radiopaque marker that is secured relative to the electrocautery electrode and is spaced apart from the first radiopaque marker. In some cases, the one or more radiopaque markers may include a pair of elongate radiopaque markers that are circumferentially spaced 180 degrees apart from each other.

In some instances, a medical device is adapted for lacerating valve leaflets. The medical device includes an elongate shaft and an inflatable balloon that is secured to the elongate shaft. The inflatable balloon has an outer surface and includes a deflated configuration and an inflated configuration. An electrocautery electrode is secured relative to the outer surface of the inflatable balloon. A radiopaque marker is disposed relative to the electrocautery electrode in order to indicate relative rotational alignment of the electrocautery electrode. In some cases, the medical device may further include a second radiopaque marker.

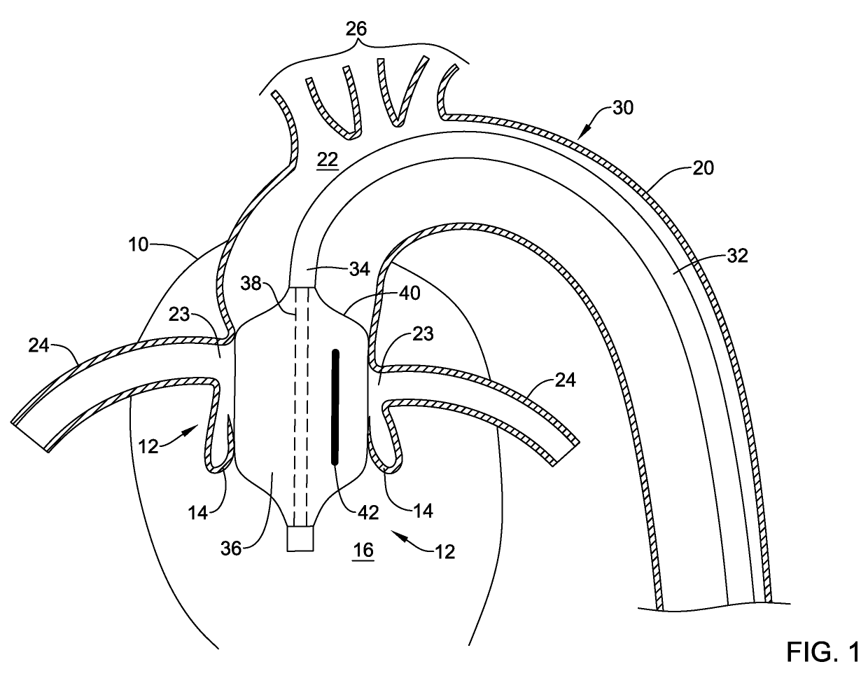

FIG. 1 is a schematic partial cut-away view of a portion of a patient’s heart 10 including an aortic valve 12 having native valve leaflets 14 disposed within and/or extending from a native valve annulus, a left ventricle 16, and certain connected vasculature, such as an aorta 20 connected to the aortic valve 12 of the patient’s heart 10 by an aortic arch 22 and an ascending aorta, the coronary ostia 23 of the coronary arteries 24, which extend from the aortic sinuses and/or the ascending aorta, and other large arteries 26 (e.g., subclavian and/or carotid arteries, etc.) that extend from the aortic arch 22 to important internal organs. While the aortic valve 12 includes a total of three native valve leaflets 14, only two are visible in the illustrated cutaway view. For the purpose of this disclosure, the discussion herein is directed toward treating the aortic valve 12 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

As shown in FIG. 1, a medical device 30 has been advanced through the aorta 20 and through the aortic arch 22 to a position in which the medical device 30 extends through the native valve leaflets 14. The medical device 30 includes an elongate shaft 32 that extends proximally from a distal region 34. An inflatable balloon 36 is secured to the distal region 34. In some cases, the elongate shaft 32 may include a shaft extension 38 that extends through an interior of the inflatable balloon 36. In some cases, the inflatable balloon 36 may be considered as having an outer surface 40. In some cases, an expandable framework may be included in place of the inflatable balloon 36. The inflatable balloon 36 may be considered as being movable between a collapsed or deflated configuration and an expanded or inflated configuration, as shown for example in FIG. 1. While the medical device 30 is shown compressing the native valve leaflets 14, it will be appreciated that the medical device 30 may also be used to compress artificial valve leaflets in a previously implanted replacement heart valve before implanting a new replacement heart valve within the previously implanted replacement heart valve. Some non-limiting examples of a replacement heart valves with which the medical device 30 may be utilized include the ACURATE NEO2™, the ACURATE PRIME™, and/or family members thereof from Boston Scientific of Marlborough, MA, USA.

Once the native valve leaflets 14 (or artificial valve leaflets) have been compressed, the leaflets may be lacerated using the medical device 30. In some cases, the medical device 30 may include an electrocautery electrode 42 that is adapted to utilize RF (radiofrequency) energy to lacerate a leaflet such as one of the native valve leaflets 14 or a replacement valve leaflet. In some cases, the electrocautery electrode 42 may extend axially along the outer surface 40 of the inflatable balloon 36. In some cases, the electrocautery electrode 42 may have a length that is sufficient to extend from a top of a native valve leaflet 14 (or top of a replacement valve leaflet) to a bottom of the native valve leaflet 14 (or the bottom of the replacement valve leaflet) such that the electrocautery electrode 42 is able to lacerate the native valve leaflet 14 (or the replacement valve leaflet) at once, without requiring translation of the electrocautery electrode 42.

The aorta 12 includes three native valve leaflets 14. In some cases, there is a desire to lacerate one of the valve leaflets 14 at a position that corresponds to where one of the coronary ostia 23 are positioned. Accordingly, it can be beneficial to be able to determine the rotational position of the electrocautery electrode 42 relative to the valve leaflet 14 that is desired to be lacerated. In some cases, the medical device 30 may include one or more radiopaque markers that, when viewed under fluoroscopy, can aid in the determination of the relative rotational position of the inflatable balloon 36, and hence the relative rotational position of the electrocautery electrode 42. When the inflatable balloon 36 is not in the correct rotational position relative to the anatomy, the electrocautery electrode 42 may not be appropriately positioned to lacerate a particular one of the native valve leaflets 14 (or a replacement valve leaflet). When the inflatable balloon 36 is in the correct rotational position relative to the anatomy, the electrocautery electrode 42 is more likely to be appropriately positioned to lacerate the particular one of the native valve leaflets 14 (or the replacement valve leaflet). Radiopaque markers may be formed from any desirable material that is visible under fluoroscopy. Examples of suitable materials are listed below.

Radiopaque markers may be placed in the inflatable balloon 36. Radiopaque markers may be placed on the outer surface 40 of the inflatable balloon 36. In some cases, radiopaque markers may be applied to the outer surface 40 of the inflatable balloon 36 via thermal deposition, printing, adhesive bonding, casting, molding and other processes. In some cases, the outer surface 40 of the inflatable balloon 36 may undergo surface preparation processes prior to applying the radiopaque markers. Examples of suitable surface preparation processes include but are not limited to laser ablation, plasma treatment and etching.

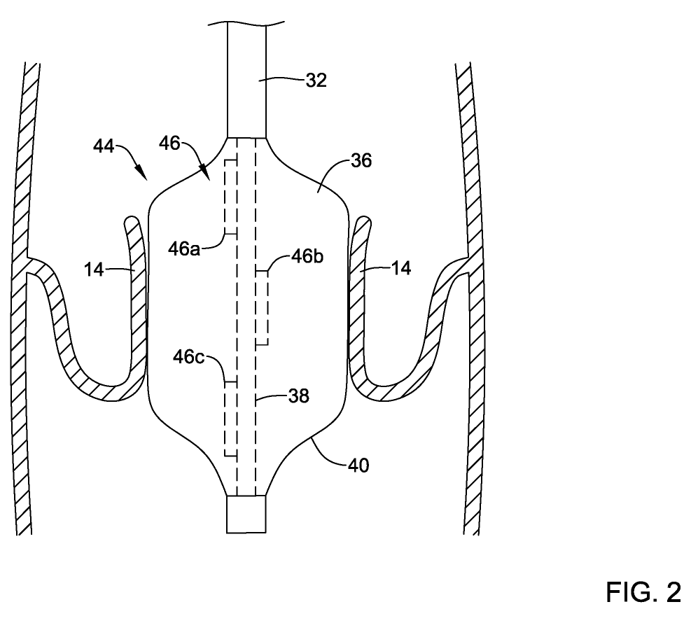

FIG. 2 shows an illustrative medical device 44 disposed between the native valve leaflets 14. The medical device 44 includes the inflatable balloon 36 extending distally from the elongate shaft 32. In some cases, as shown, several radiopaque markers 46, including a radiopaque marker 46a, a radiopaque marker 46b, and a radiopaque marker 46c, are shown disposed along the shaft extension 38. As shown, the radiopaque marker 46a and the radiopaque marker 46c are shown disposed on a left (in the illustrated orientation) side of the shaft extension 38 and the radiopaque marker 46b is shown disposed along a right (on the illustrated orientation) side of the shaft extension 38. Under fluoroscopy, this LEFT-RIGHT-LEFT pattern indicates a particular rotational position of the inflatable balloon 36. If the inflatable balloon 36 was rotated 180 degrees, the radiopaque markers 46 would exhibit a RIGHT-LEFT-RIGHT pattern, indicating that the inflatable balloon 36 has been rotated 180 degrees relative to the illustrated orientation. While each of the radiopaque markers 46 are shown as being the same length, this is not required. Some of the radiopaque markers 46 may be longer or shorter than others of the radiopaque markers 46. The electrocautery electrode 42 is not shown in FIG. 2.

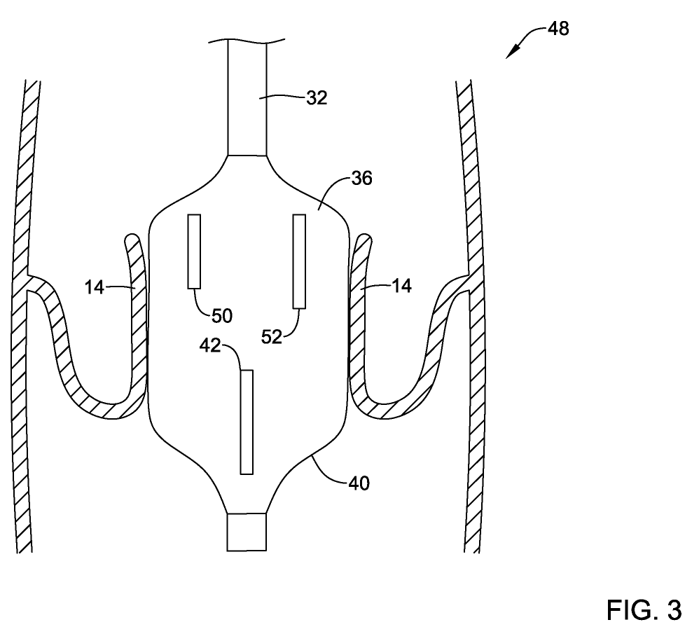

FIG. 3 shows an illustrative medical device 48 disposed between the native valve leaflets 14. The medical device 48 includes the inflatable balloon 36 extending distally from the elongate shaft 32. For clarity, the shaft extension 38 is not shown in FIG. 3. The electrocautery electrode 42 is disposed on the outer surface 40 of the inflatable balloon 36. While it is appreciated that there would be an electrical connection extending through the elongate shaft 32 and across the inflatable balloon 36 to provide RF energy to the electrocautery electrode 42, this electrical connection is not shown. The medical device 48 includes a first radiopaque marker 50 and a second radiopaque marker 52. In some cases, the first radiopaque marker 50 and the second radiopaque marker 52 may be disposed on the outer surface 40 of the inflatable balloon 36. In some cases, the first radiopaque marker 50 and the second radiopaque marker 52 may be disposed between layers forming the inflatable balloon 36.

While the first radiopaque marker 50 and the second radiopaque marker 52 are shown as being parallel or at least substantially parallel with the electrocautery electrode 42, defined as within twenty percent of parallel, this is not required in all case. For example, one or more of the first radiopaque marker 50 and the second radiopaque marker 52 may be disposed at an angle relative to the electrocautery electrode 42. In some cases, the medical device 48 may include additional radiopaque markers. In some cases, the electrocautery electrode 42 itself may be at least partially visible during fluoroscopy.

As shown, the first radiopaque marker 50 is shorter than the second radiopaque marker 52. As a result, the relative rotational position of the inflatable balloon 36, and hence the electrocautery electrode 42, may be ascertained during fluoroscopy. In some cases, the first radiopaque marker 50 may have a length that is at least twenty to thirty percent, or twenty five, percent shorter than the length of the second radiopaque marker 52 so that that a difference in length between the first radiopaque marker 50 and the second radiopaque marker 52 is detectable under fluoroscopy. Under fluoroscopy, with the inflatable balloon 36 positioned as shown, with the electrocautery electrode 42 disposed on a front (in the illustrated orientation) of the inflatable balloon 36, the shorter first radiopaque marker 50 will appear to be to the left of the electrocautery electrode 42 while the longer second radiopaque marker 52 will be visible to the right of the electrocautery electrode 42. If the medical device 48, and hence the inflatable balloon 36, were rotated 180 degrees from the illustrated orientation, meaning that the electrocautery electrode 42 would be positioned on a back (in the illustrated orientation) of the inflatable balloon 36, then the shorter first radiopaque marker 50 will appear to be to the right of the electrocautery electrode 42 while the longer second radiopaque marker 52 will appear to be to the left of the electrocautery electrode 42. Rotating the medical device 48, and hence the inflatable balloon 36, a different amount will result in the apparent positions of the first radiopaque marker 50 and the second radiopaque marker 52 changing accordingly.

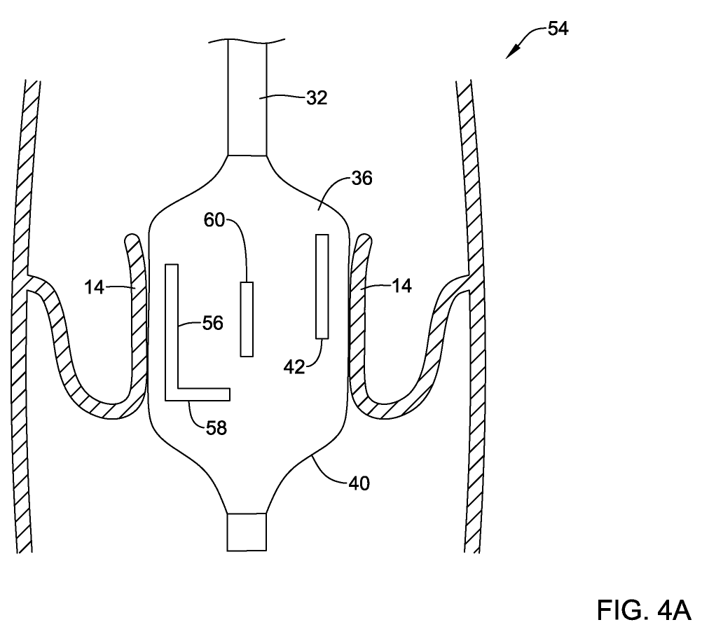

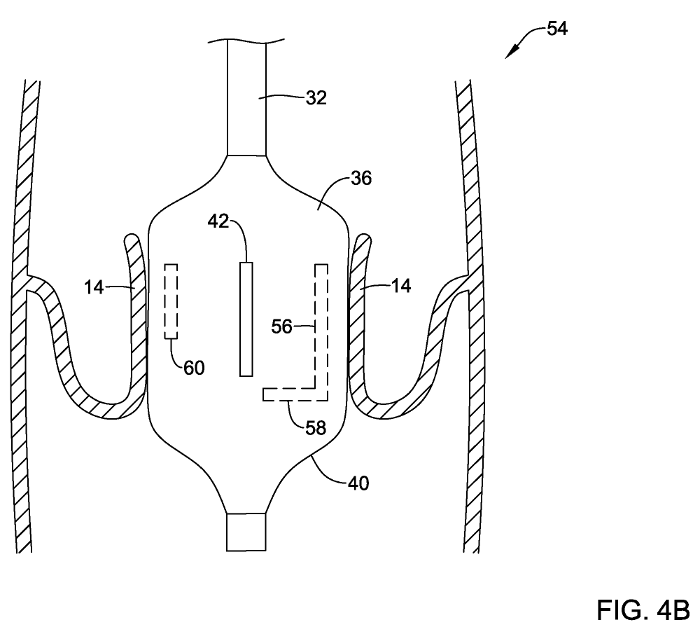

FIGS. 4A and 4B show an illustrative medical device 54 disposed between the native valve leaflets 14. The medical device 54 includes the inflatable balloon 36 extending distally from the elongate shaft 32. For clarity, the shaft extension 38 is not shown in FIGS. 4A and 4B. The electrocautery electrode 42 is disposed on the outer surface 40 of the inflatable balloon 36. While it is appreciated that there would be an electrical connection extending through the elongate shaft 32 and across the inflatable balloon 36 to provide RF energy to the electrocautery electrode 42, this electrical connection is not shown. For FIGS. 4A and 4B, it is assumed that there is a desire to lacerate a native valve leaflet 14 (or a replacement valve leaflet) that is positioned between the two visible native valve leaflets 14 (or the replacement valve leaflets). The third native valve leaflet 14 may be positioned in front of the page, for example. In FIG. 4A, the electrocautery electrode 42 is not appropriately positioned to be able to lacerate the unseen third native valve leaflet 14 (or the unseen replacement valve leaflet). In FIG. 4B, the medical device 54, and hence the inflatable balloon 36, has been rotated such that the electrocautery electrode 42 is appropriately positioned to lacerate the unseen third valve leaflet 14 (or the unseen replacement valve leaflet).

The relative position of the inflatable balloon 36 may be determined via fluoroscopy by identifying the relative locations of radiopaque markers on or in the inflatable balloon 36. As seen in FIG. 4A, a first radiopaque marker segment 56 extends parallel or at least substantially parallel with the electrocautery electrode 42 and a second radiopaque marker segment 58 that is positioned next to, or even in contact with, the first radiopaque marker segment 56. The second radiopaque marker segment 58 extends orthogonally, or at least substantially orthogonally to the first radiopaque marker segment 56, and thus orthogonally, or at least substantially orthogonally, to the electrocautery electrode 42. The medical device 54 also includes a shorter radiopaque marker 60. In comparing FIG. 4A with FIG. 4B, the first radiopaque marker segment 56 and the second radiopaque marker segment 58 combine to form the appearance of an “L” when the medical device 54 is not appropriately positioned, and form the appearance of a “J” when the medical device 54 is appropriately positioned.

In some cases, the first radiopaque marker segment 56, the second radiopaque marker segment 58, and the radiopaque marker 60 may be disposed on the outer surface 40 of the inflatable balloon 36. In some cases, the first radiopaque marker segment 56, the second radiopaque marker segment 58, and the radiopaque marker 60 may be disposed between layers forming the inflatable balloon 36. In some cases, the radiopaque marker 60 may be excluded. In some cases, the inflatable balloon 36 may include additional radiopaque markers.

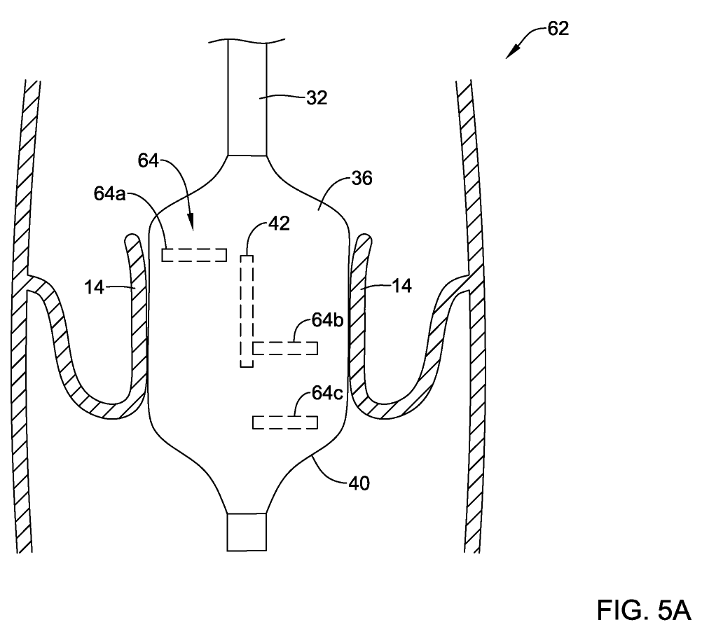

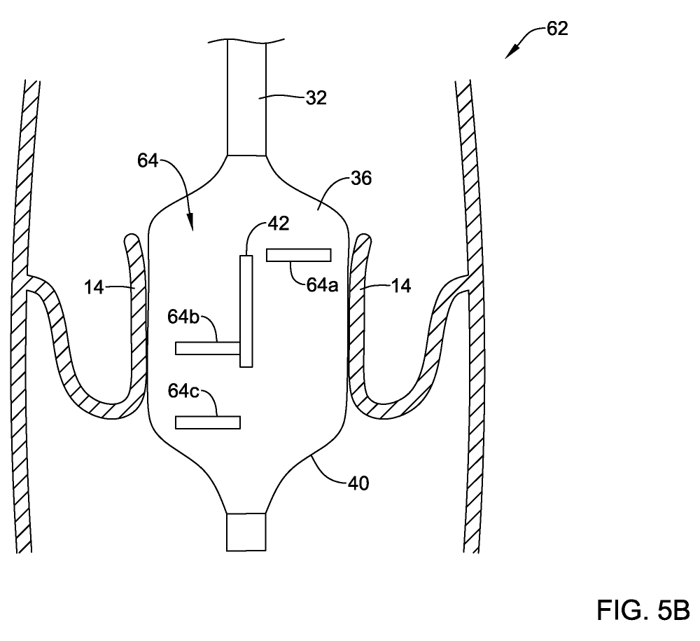

FIGS. 5A and 5B show an illustrative medical device 62 disposed between the native valve leaflets 14. The medical device 62 includes the inflatable balloon 36 extending distally from the elongate shaft 32. For clarity, the shaft extension 38 is not shown in FIGS. 5A and 5B. The electrocautery electrode 42 is disposed on the outer surface 40 of the inflatable balloon 36. While it is appreciated that there would be an electrical connection extending through the elongate shaft 32 and across the inflatable balloon 36 to provide RF energy to the electrocautery electrode 42, this electrical connection is not shown. For FIGS. 5A and 5B, it is assumed that there is a desire to lacerate a native valve leaflet 14 (or a replacement valve leaflet) that is positioned between the two visible native valve leaflets 14 (or the replacement valve leaflets). The third native valve leaflet 14 may be positioned in front of the page, for example.

In FIG. 5A, the electrocautery electrode 42 is not appropriately positioned to be able to lacerate the unseen third native valve leaflet 14 (or the unseen replacement valve leaflet). In FIG. 5B, the medical device 62, and hence the inflatable balloon 36, has been rotated such that the electrocautery electrode 42 is appropriately positioned to lacerate the unseen third valve leaflet 14 (or the unseen replacement valve leaflet). It is assumed, for example, that in FIG. 5A, the electrocautery electrode 42 is disposed on a back (in the illustrated orientation) side of the inflatable balloon 36 while in FIG. 5B the electrocautery electrode 42 is disposed on a front (in the illustrated orientation) side of the inflatable balloon 36. The relative position of the inflatable balloon 36 may be determined via fluoroscopy by identifying the relative locations of radiopaque markers on or in the inflatable balloon 36.

The medical device 62 includes several circumferentially extending radiopaque markers 64 that are orthogonal or at least substantially orthogonal, to the electrocautery electrode 42. In some cases, the radiopaque markers 64 may be disposed on the outer surface 40 of the inflatable balloon 36. In some cases, the radiopaque markers 64 may be disposed between layers forming the inflatable balloon 36. The radiopaque markers 64 include a first radiopaque marker 64a that is disposed to a left (in the illustrated orientation) side of the electrocautery electrode 42. A second radiopaque marker 64b is disposed to a right (in the illustrated orientation) side of the electrocautery electrode 42. A third radiopaque marker 64c is disposed to a right (in the illustrated orientation) side of the electrocautery electrode 42. Each of the radiopaque markers 64 are axially spaced from each other. In FIG. 5A, which shows the electrocautery electrode 42 disposed on a back side of the inflatable balloon 36, the radiopaque markers 64 form a LEFT-RIGHT-RIGHT pattern. In FIG. 5B, which shows the electrocautery electrode 42 disposed on a front side of the inflatable balloon 36, the radiopaque markers 64 form a RIGHT-LEFT-LEFT pattern, indicating that the electrocautery electrode 42 is appropriately positioned.

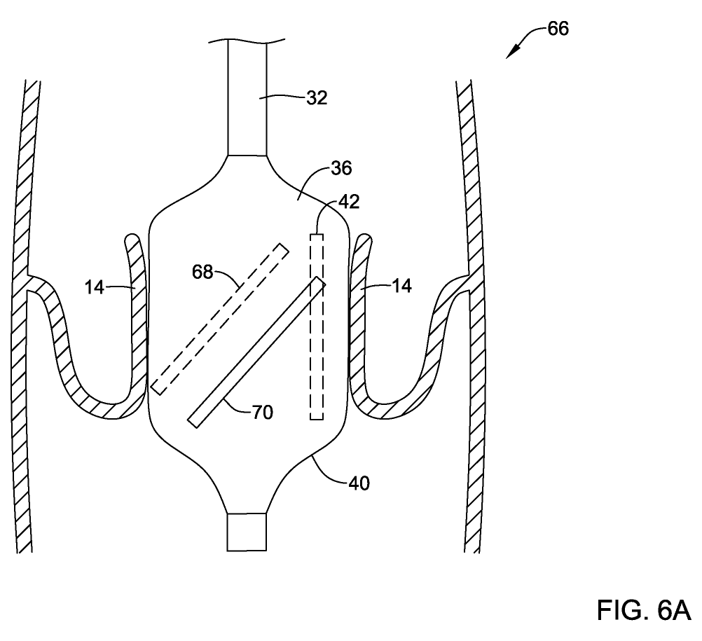

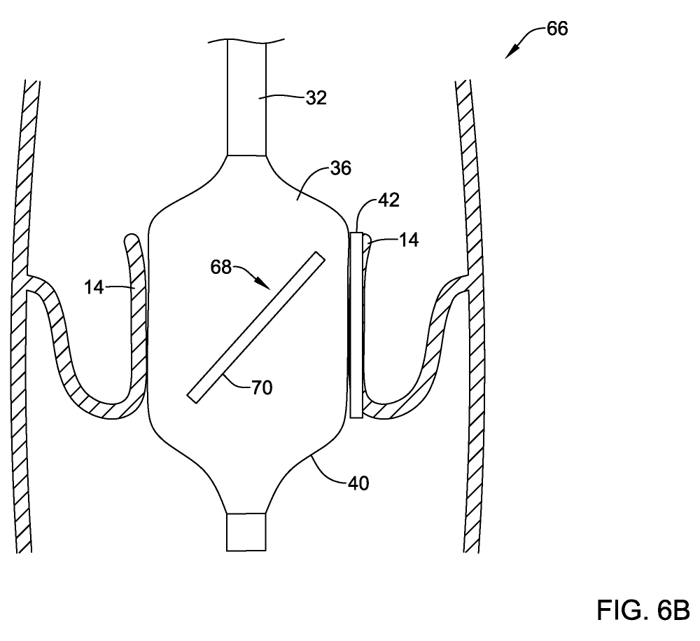

FIGS. 6A and 6B show an illustrative medical device 66 disposed between the native valve leaflets 14. The medical device 66 includes the inflatable balloon 36 extending distally from the elongate shaft 32. For clarity, the shaft extension 38 is not shown in FIGS. 6A and 6B. The electrocautery electrode 42 is disposed on the outer surface 40 of the inflatable balloon 36. While it is appreciated that there would be an electrical connection extending through the elongate shaft 32 and across the inflatable balloon 36 to provide RF energy to the electrocautery electrode 42, this electrical connection is not shown. For FIGS. 6A and 6B, it is assumed that there is a desire to lacerate a native valve leaflet 14 (or a replacement valve leaflet) that is positioned to the right side (in the illustrated orientation) of the inflatable balloon 36.

In FIG. 6A, the electrocautery electrode 42 is not appropriately positioned to be able to lacerate the unseen third native valve leaflet 14 (or the unseen replacement valve leaflet). In FIG. 5B, the medical device 62, and hence the inflatable balloon 36, has been rotated such that the electrocautery electrode 42 is appropriately positioned to lacerate the unseen third valve leaflet 14 (or the unseen replacement valve leaflet). It is assumed, for example, that in FIG. 6A, the electrocautery electrode 42 is disposed on a back (in the illustrated orientation) side of the inflatable balloon 36 while in FIG. 6B the electrocautery electrode 42 is disposed on a right (in the illustrated orientation) side of the inflatable balloon 36. The relative position of the inflatable balloon 36 may be determined via fluoroscopy by identifying the relative locations of radiopaque markers on or in the inflatable balloon 36.

The medical device 66 includes a first radiopaque marker 68 and a second radiopaque marker 70. As shown in FIG. 6A, the first radiopaque marker 68 may be disposed on a back (in the illustrated orientation) side of the inflatable balloon 36 while the second radiopaque marker 70 may be disposed on a front (in the illustrated orientation) side of the inflatable balloon 36. As shown in FIG. 6A, the first radiopaque marker 68 and the second radiopaque marker 70 do not appear to be very far apart. When the medical device 66, and hence the inflatable balloon 36 is rotated a small distance, the second radiopaque marker 70 blocks the view of the first radiopaque marker 68, as shown in FIG. 6B. When the first radiopaque marker 68 and the second radiopaque marker 70 appear as a single line during fluoroscopy, the electrocautery electrode 42 is properly positioned next to the native valve leaflet 14 to the right of the inflatable balloon 36. In some cases, the first radiopaque marker 68 and the second radiopaque marker 70 may appear as a single line that is wider than either of the first radiopaque marker 68 or the second radiopaque marker 70. In some cases, the first radiopaque marker 68 and the second radiopaque marker 70 may be disposed on the outer surface 40 of the inflatable balloon 36. In some cases, the first radiopaque marker 68 and the second radiopaque marker 70 may be disposed between layers forming the inflatable balloon 36.

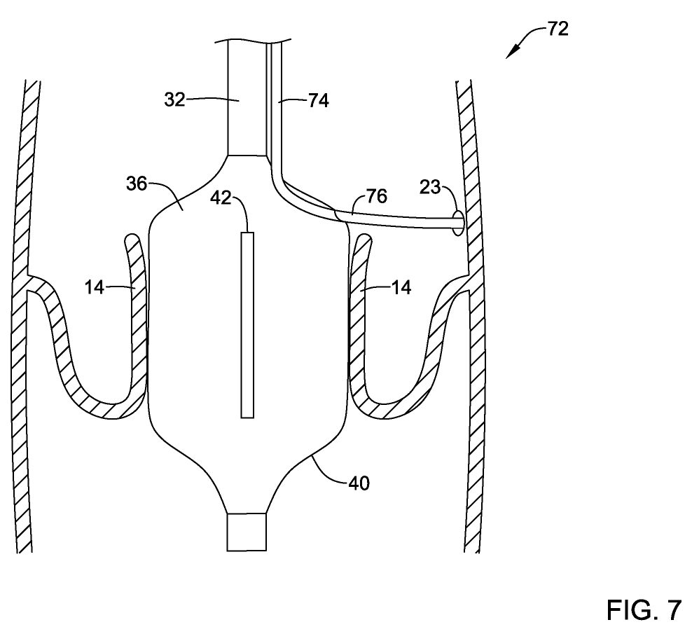

FIG. 7 shows an illustrative medical device 72 that is disposed between the native valve leaflets 14. The medical device 72 includes the inflatable balloon 36 extending distally from the elongate shaft 32. For clarity, the shaft extension 38 is not shown in FIG. 7. The electrocautery electrode 42 is disposed on the outer surface 40 of the inflatable balloon 36. While it is appreciated that there would be an electrical connection extending through the elongate shaft 32 and across the inflatable balloon 36 to provide RF energy to the electrocautery electrode 42, this electrical connection is not shown. While no radiopaque markers are shown, it will be appreciated that the medical device 72 may include any manner and combination of radiopaque markers such as the radiopaque markers 46, 50, 52, 56, 58, 60, 64, 68, and 70 as shown herein with respect to the other drawings. The medical device 72 includes a lumen 74 that extends along the elongate shaft 32 and provides a contrast fluid (shown schematically as 76) that may be used to help locate one of the coronary ostia 23. In some cases, the lumen 74 may be disposed within the elongate shaft 32. Any suitable contrast fluid 76 may be used, including iodine-based agents commonly used in X-ray and CT imaging.

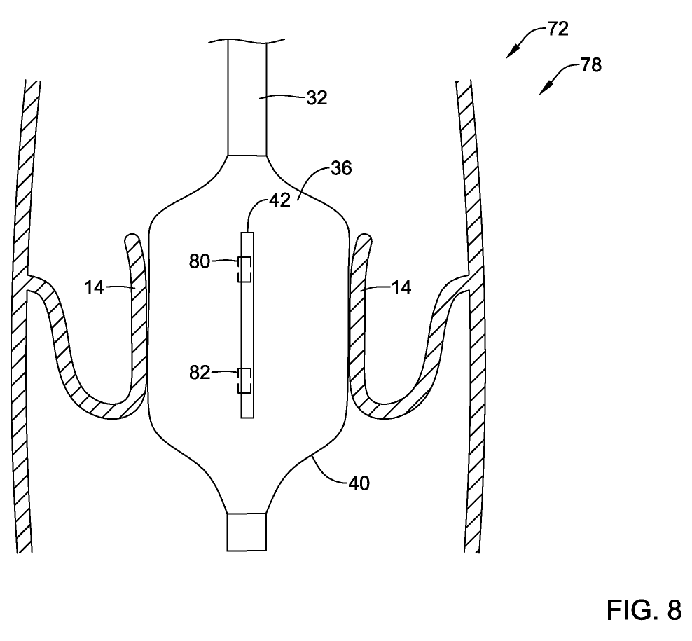

FIG. 8 shows an illustrative medical device 78 that is disposed between the native valve leaflets 14. The medical device 78 includes the inflatable balloon 36 extending distally from the elongate shaft 32. For clarity, the shaft extension 38 is not shown in FIG. 8. The electrocautery electrode 42 is disposed on the outer surface 40 of the inflatable balloon 36. While it is appreciated that there would be an electrical connection extending through the elongate shaft 32 and across the inflatable balloon 36 to provide RF energy to the electrocautery electrode 42, this electrical connection is not shown. While not shown, the medical device 78 may include any manner and combination of radiopaque markers such as the radiopaque markers 46, 50, 52, 56, 58, 60, 64, 68, and 70 as shown herein with respect to the other drawings. In some cases, the medical device 78 may alternatively or additionally include a first radiopaque marker 80 that is secured to the electrocautery electrode 42 and a second radiopaque marker 82 that is secured to the electrocautery electrode 42. In some cases, the first radiopaque marker 80 and the second radiopaque marker 82 may be spaced apart a distance that is about equal to an average height of a native valve leaflet from top to bottom. In some cases, the first radiopaque marker 80 and the second radiopaque marker 82 may help with axial positioning of the medical device 78, and hence the inflatable balloon 36, relative to the native valve leaflet 14 (or replacement valve leaflet) that an operator wants to lacerate.

In some cases, radiopaque markers such as one or more of the radiopaque markers 46, 50, 52, 56, 58, 60, 64, 68, 70, 80 and 82 may not be formed of a radiopaque material, but may instead represent an absence of radiopaque material. As an example, the entirety of the inflatable balloon 36 may be filled with a fluid that is visible under fluoroscopy. The markers may instead be parts or spaces within the inflatable balloon 36 that are free of such material. So, rather than the inflatable balloon 36 appearing clear or mostly clear under fluoroscopy, and the radiopaque markers (and possibly the electrocautery electrode 42) appearing as dark lines under fluoroscopy, the markers would appear as lighter spots or shapes while the rest of the inflatable balloon 36 would appear dark. Examples of segmented balloons that may be used to create “radiopaque” markers that exhibit an absence of radiopaque material may be found in U.S. 2023/0363786 and U.S. 2024/0050715, each of which are herein incorporated by reference in their entirety.

The materials that can be used for the various components of the devices and various elements thereof disclosed herein may include those commonly associated with medical devices. In some instances, the medical devices, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem) or Vestamid L21® available from Evonik Industries, elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the medical devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten, tungsten alloy, tungsten/polymer blends, conductive inks, iodine, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the medical devices and/or other elements disclosed herein. For example, the medical devices, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the medical devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure’s scope is, of course, defined in the language in which the appended claims are expressed.

What is claimed is:

1. A medical device for lacerating valve leaflets, the medical device comprising:
an elongate shaft;
an inflatable balloon secured to the elongate shaft, the inflatable balloon having an outer surface and including a deflated configuration and an inflated configuration;
an electrocautery electrode secured relative to the outer surface of the inflatable balloon; and
one or more radiopaque markers disposed relative to the electrocautery electrode, the one or radiopaque markers adapted to indicate relative rotational alignment of the electrocautery electrode.

2. The medical device of claim 1, wherein the one or more radiopaque markers are disposed on the outer surface of the inflatable balloon.

3. The medical device of claim 1, wherein the one or more radiopaque markers are disposed within the inflatable balloon.

4. The medical device of claim 3, wherein the elongate shaft includes a shaft portion extending through an interior of the inflatable balloon and the one or more radiopaque markers are secured relative to the shaft portion.

5. The medical device of claim 1, wherein the one or more radiopaque markers include one or more radiopaque markers that are at least substantially parallel with the electrocautery electrode.

6. The medical device of claim 1, wherein the one or more radiopaque markers include one or more radiopaque markers that are at least substantially orthogonal to the electrocautery electrode.

7. The medical device of claim 1, wherein the one or more radiopaque markers include one or more radiopaque marker segments that are at least substantially parallel with the electrocautery electrode and one or more radiopaque marker segments that are at least substantially perpendicular to the electrocautery electrode.

8. The medical device of claim 1, wherein the electrocautery electrode is adapted to be visible during fluoroscopy.

9. The medical device of claim 1, wherein the one or more radiopaque markers comprise a first radiopaque marker secured relative to a first end of the electrocautery electrode and a second radiopaque marker secured relative to a second end of the electrocautery electrode.

10. The medical device of claim 1, wherein the one or more radiopaque markers comprise a pair of elongate radiopaque markers that are circumferentially spaced 180 degrees apart from each other.

11. The medical device of claim 1, wherein the elongate shaft further comprises a lumen for injecting contrast media.

12. A medical device for lacerating valve leaflets, the medical device comprising:
an inflatable balloon having an outer surface and including a deflated configuration and an inflated configuration;
an electrocautery electrode secured relative to the inflatable balloon; and
one or more radiopaque markers secured relative to the inflatable balloon.

13. The medical device of claim 12, wherein the one or more radiopaque markers are disposed on the outer surface of the inflatable balloon.

14. The medical device of claim 12, wherein the one or more radiopaque markers are at least substantially parallel with the electrocautery electrode.

15. The medical device of claim 12, wherein the one or more radiopaque markers are at least substantially orthogonal to the electrocautery electrode.

16. The medical device of claim 12, wherein some of the one or more radiopaque markers are at least substantially parallel with the electrocautery electrode and some of the one or more radiopaque marker segments are at least substantially perpendicular to the electrocautery electrode.

17. The medical device of claim 12, wherein the one or more radiopaque markers comprise a first radiopaque marker secured relative to the electrocautery electrode and a second radiopaque secured relative to the electrocautery electrode and spaced apart from the first radiopaque marker.

18. The medical device of claim 12, wherein the one or more radiopaque markers comprise a pair of elongate radiopaque markers that are circumferentially spaced 180 degrees apart from each other.

19. A medical device for lacerating valve leaflets, the medical device comprising:
an elongate shaft;
an inflatable balloon secured to the elongate shaft, the inflatable balloon having an outer surface and including a deflated configuration and an inflated configuration;
an electrocautery electrode secured relative to the outer surface of the inflatable balloon; and
a radiopaque marker disposed relative to the electrocautery electrode in order to indicate relative rotational alignment of the electrocautery electrode.

20. The medical device of claim 19, further comprising a second radiopaque marker.
